(19) United States
(12) Patent Application Publication
(10) Pub. No.: US 2003/0077612 A1
(43) Pub. Date: Apr. 24, 2003

(54) Method

(76) Inventor: Jonas Ekblom, Uppsala (SE)

Correspondence Address: JANIS K. FRASER, PH.D., Fish & Richardson P.C., 225 Franklin Street, Boston, MA, 02110-2804, US

(21) Appl. No.: 10/116,265

(22) Filed: Apr. 4, 2002

Related U.S. Application Data
(60) Provisional application No. 60/281,384, filed on Apr. 5, 2001.

(30) Foreign Application Priority Data
Apr. 5, 2001 (SE) 0101218-6

(51) Int. Cl. C12Q 1/68; G06F 19/00; G01N 33/48; G01N 33/50

(52) U.S. Cl. 435/6; 702/20; 435/455

(57) ABSTRACT

The invention concerns a method for identifying RNA-binding molecules, comprising the steps of: predicting the structure of an RNA-fragment by an in silico method, choosing a suitable predicted RNA-fragment, synthesizing the cDNA-fragment corresponding to the predicted RNA-fragment, inserting the cDNA-fragment in the upstream proximity of a reporter assay gene, which reporter assay gene produces a signal upon translation, thereby forming a reporter construct, and performing a reporter gene assay, which assay monitors the interaction between a molecule to be tested for RNA-binding and the RNA-fragment of the reporter construct. Furthermore, the invention relates to the use of specific RNA-fragments in the method of the invention.

### CROSS REFERENCE TO RELATED APPLICATIONS

This application claims priority from Swedish Patent Application No. 0101218-6, filed Apr. 5, 2001, and U.S. Provisional Patent Application Serial No. 60/281,384, filed Apr. 5, 2001. These applications are incorporated herein by reference in their entirety.

### TECHNICAL FIELD

The invention relates to a method for identifying RNA-binding molecules, as well as the use of specific RNA-molecules in the method.

### BACKGROUND

RNA (ribonucleic acid) was earlier seen as a linear information-carrying molecule, having no specific structural properties. Gradually it has been understood that RNA may possess complex and strong three-dimensional structures, such as hairpins. Moreover, it has been shown that some structural motifs may bind various small molecules with high affinity. Furthermore, it has been shown that strong secondary RNA structures lower the translational efficacy (Werstuck, G. & Green, M. R. (1998) Science 282: 296-298).

Recently, it was proposed to use RNA as a drug target (Ecker D J & Griffey R H (1999) Drug Discovery Today 4: 420-430) because of its small-molecule binding properties at strong three-dimensional internal structures. Moreover, methods have been presented for finding molecules binding to interesting RNA-structures, involving the steps of (a) predicting in silico one or several RNA-structures from given sequences, (b) purifying a chosen RNA-structure, and (c) monitoring binding to small molecules by mass spectrometry (Hofstadler & Griffey, (2000) Curr. Opin. Drug Discovery & Development 3: 423-431). However, this biochemical analysis method has drawbacks in the respect that the interactions are not studied in a physiological context, i.e. in a living cell or an organism. Therefore, molecules found by this method may not be fully applicable in the body, e.g. they may not be membrane permeable.

Accordingly, there is a need for screening methods for interactions between small-molecules and RNA-structures, limiting the drawbacks mentioned above.

The object of the invention is to provide a method, which satisfies the need set out above.

### SUMMARY OF THE INVENTION

This object is fulfilled by a method for identifying RNA-binding molecules, comprising the steps of:

(a) predicting the structure of an RNA-fragment, preferably by an in silico method;

(b) choosing a suitable predicted RNA-fragment of step (a), which RNA-fragment comprises at least one individual stem;

(c) synthesizing the DNA-fragment corresponding to the RNA-fragment of step (b);

(d) inserting the DNA-fragment of step (b) in the upstream proximity of a reporter assay gene, which reporter assay gene produces a signal upon translation, thereby forming a reporter construct; and

(e) performing a reporter gene assay, which assay monitors the interaction between a molecule to be tested for RNA-binding and the RNA-fragment of the reporter construct.

Hereby, the translational inhibition or potentiation effect, caused by strong RNA-structures, is used to screen for RNA-binding drug molecules. The in silico prediction according to step (a) above is preferably performed by the “Zuker & Mathewns” algorithm or the “van Batenburg” algorithm (see below for references). Moreover, the identification is preferably performed in living cells, resulting in that the substances have normal membrane permeability, which is advantageous from a pharmacological viewpoint. Furthermore, the free Gibbs energy for an individual stem should be lower than −5 kcal/mol, preferably lower than −10 kcal/mol. These parameters can be calculated by the above prediction algorithms. Maximal strength of a stem loop is obtained if all nucleotides are involved in base pairing, i.e. the ratio of the number of nucleotides per base pairing is 2. Accordingly, the ratio of nucleotides per base pairing in any given structure for drug targeting should be as low as possible; ideally, lower than 4. The length of the stem (the sequence) should preferably be shorter than 100 nucleotides. Specifically, the reporter assay gene may be a luciferase gene, thereby providing an easily detectable method.

In a preferred embodiment, the reporter gene assay comprises the steps of:

(f) transfecting cells with the reporter construct;

(g) culturing the transfected cells of step (f);

(h) adding a molecule to be tested for RNA-binding to the cultured cells; and

(i) monitoring the reporter signal, which signal indicates the interaction status between the molecule to be tested for RNA-binding and the RNA-fragment.

Furthermore, the invention relates to the use of any one of the RNA-sequences SEQ ID NO:2, SEQ ID NO:4, SEQ ID NO:6, SEQ ID NO:8, SEQ ID NO: 10, SEQ ID NO:12, SEQ ID NO:14, SEQ ID NO:16 or SEQ ID NO:18, corresponding to the target region, and more specifically any one of the RNA-sequences SEQ ID NO: 1, SEQ ID NO:3, SEQ ID NO:5, SEQ ID NO:7, SEQ ID NO:9, SEQ ID NO:11, SEQ ID NO:13, SEQ ID NO: 15 or SEQ ID NO: 17, corresponding to the 5′-UTR-region, for identifying small molecules by use of the method described above.

Accordingly, the invention provides a method being performed in living cells or extracts of living cells (in vitro translation), and which method due to its nature is rapid to use for screening for the binding of a large number of small molecules to a specific RNA-structure. In addition, this concept has a number of advantages as compared to classical drug discovery: i) it is possible to modify target genes from any gene family (at protein level only a few protein classes are considered “targetable”), ii) the concept eliminates selectivity issues since the RNA target may be chosen in a region of transcript having low homology to other sequences, and iii) both down- and up-regulation may be possible.

Unless otherwise defined, all technical and scientific terms used herein have the same meaning as commonly understood by those of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of a conflict in terminology, the present specification will control. In addition, the described materials and methods are illustrative only and are not intended to be limiting.

Other features and advantages of the invention will be apparent from the following detailed description and the claims.

### BRIEF DESCRIPTION OF THE DRAWINGS

FIG. 1 depicts a vector containing the sequence of SEQ ID NO:19 inserted upstream of a luciferase reporter gene.

FIGS. 2 and 3 depict chemical entities that had specific effects on mRNA having the “mLPL-structure.”

### DEFINITIONS

By “molecules binding to RNA” is meant any molecule binding to, and thereby stabilizing, a certain RNA-structure.

By an “RNA-fragment” is meant any stretch or part of an RNA sequence.

By an “individual stem” is meant a structure in an RNA-molecule, in which at least the first and the last nucleotides of a sequence interact through base-pair interaction. For example, a so-called hairpin may serve as an example of an individual stem. Ideally, a large fraction of the nucleotides in an individual stem are involved in base pairing.

By “the free Gibbs energy for an individual stem” is meant the energy that the particular stem adds to the energy of the total structure.

By “predicting by in silico methods” is meant to use some kind of molecular modeling algorithm in order to achieve a modeled structure of a molecule.

By “suitable predicted” RNA-fragment is meant an RNA-fragment exhibiting structural features indicating that it has a good potential for binding of another molecule.

By “upstream proximity of a reporter assay gene” is meant a position 5′ of the reporter assay gene, and close to the reporter assay gene, preferably in a so-called 5′-untranslated region.

By a “reporter assay” is meant any assay producing a signal upon translation, or in the absence of translation, of the reporter assay gene transcript.

By “indicating interaction status” is meant that the possible binding between a molecule to be tested for RNA-binding and the RNA-fragment can be determined.

By “non-peptide and/or non-nucleotide molecules” are meant large molecules, not being entirely constructed of amino acids or nucleic acids in a sequence.

### DISCLOSURE OF THE INVENTION

In a first aspect, the invention provides a method for identifying RNA-binding molecules, as set out above. The several steps of the method may be varied in several ways. However, the main characteristics of the inventive method are (a) that the RNA-structures are predicted by an in silico prediction method, such as the methods of van Batenberg or Zuker & Mathews, (b) that the CDNA corresponding to the predicted RNA-structure is synthesized, and (c) that a reporter assay for living cells are used to monitor the interaction between potential RNA-binding molecules and the chosen RNA-structure.

A suitable RNA structure for drug targeting according to the invention shows the following characteristics: (i) it has a sufficient stability in order to maintain its integrity within a variety of sequence contexts, i.e., it has a high stability, (ii) it is contained within a sequence fragment that is short enough to allow artificial synthesis, and (iii) it represents a sequence that is unique, in order to prevent selectivity issues. As a guiding principle, the following criteria have been used to select suitable RNA-sequences:

(a) individual stems should have free Gibbs energies lower than −5 keal/mol, preferably lower than −10 kcal/mol,

(b) individual stems should have a ratio between number of nucleotides per base pair of less than 4

(c) individual stems should be predicted to maintain their structure in a context of up to 400 nucleotides of a native sequence,

(d) stems/structures are contained within sequence fragments shorter than 100 nucleotides,

(e) primary structures (sequences) should have less than 70% homology to any other known sequence, as determined by, for instance, a BLAST homology comparison.

For the purpose of screening, a stretch of RNA is chosen that constitutes a defined sub-domain. A functional sub-domain in RNA is a fragment that, when removed from the larger RNA and studied in isolation, retains its biological/in silico shape. Accordingly, in an initial analysis a large portion of RNA may be used for computer-assisted predictions. Folds that are larger than 20 base pairs and lack bifurcations (branches) are re-analyzed with the prediction software. If such a fold is predicted to retain its structure without its larger context, it is considered a suitable target RNA structure.

A defined double-stranded cDNA fragment corresponding to a predicted structure in a specific mRNA may be synthesized artificially, typically 20-200 nucleotides long. The cDNA is synthesized with flanking overhangs corresponding to defined restriction cleavage sites, e.g. Hindlll, EcoRI, BamHl etc. Conveniently, the double-stranded synthetic cDNA may be ligated into a suitable reporter vector, preferably, into the 5′-UTR region of the reporter gene. One example of such a vector can be pGL3 control (Promega, USA), which encodes luciferase as a reporter gene (inserts may be ligated to the HindIII site). In principle, any gene encoding a detectable protein may in principle be utilized for this purpose, for instance green fluorescent protein (GFP), alkaline phosphatase, beta-galactosidase, lactamase etc. Accordingly, the “RNA-structure” will be included in the 5′-end of the reporter transcript. Small molecules that bind to such a structure and affect its translation will cause a shift in the reporter gene expression.

The plasmid construct can be transfected into virtually any mammalian cell type, for example Caco-2, COS, CHO, HEK293 etc. Moreover, it is also plausible to use insect cells or different strains of yeast. Transfection may be accomplished by several different protocols e.g., by treatment with calcium phosphate, with liposomes, or with electroporation etc. It is also foreseeable that stable cell lines can be useful for this reporter assay screening protocol. After transfection, cells can be plated into multi-well plates, commonly 96- or 384-well plates. After adhesion, different test drugs can be applied to the wells and after a defined period of exposure (typically 2-24 h) the reporter gene expression can be estimated using standard procedures. Positive hits, i.e. compounds that significantly affected expression of the reporter gene will also be assayed with cells transfected with a plasmid lacking the “RNA structure insert”, i.e. a control vector. Compounds that significantly modulate the expression of the reporter gene containing the “RNA structure insert” while having no effect on a reporter gene lacking the “RNA structure insert” will be considered as “true hits”. One may postulate that it is possible by such a screening procedure to identify compounds having specific effects, both activation ad inhibition, mediated via a defined RNA structure.

For screening purposes, appropriate host cells can be transformed with a vector having a reporter gene under the control of the RNA-fragment according to this invention. The expression of the reporter gene can be measured in the presence or absence of an agent with known activity (i.e. a standard agent) or putative activity (i.e. a “test agent” or “candidate agent”). A change in the level of expression of the reporter gene in the presence of the test agent is compared with that effected by the standard agent. In this way, active agents are identified and their relative potency in this assay determined.

A transfection assay can be a particularly useful screening assay for identifying an effective agent. In a transfection assay, a nucleic acid containing a gene such as a reporter gene that is operably linked to a suitable promoter, or an active fragment thereof, is transfected into the desired cell type. A test level of reporter gene expression is assayed in is the presence of a candidate agent and compared to a control level of expression. An effective agent is identified as an agent that results in a test level of expression that is different than a control level of reporter gene expression, which is the level of expression determined in the absence of the agent. Methods for transfecting cells and a variety of convenient reporter genes are well known in the art (see, for example, Goeddel (ed.), Methods Enzymol., Vol. 185, San Diego: Academic Press, Inc. (1990); see also Sambrook, supra).

As used herein, the term “reporter gene” means a gene encoding a gene product that can be identified using simple, inexpensive methods or reagents and that can be operably linked to the RNA-fragment of the invention, or an active fragment thereof. Reporter genes such as, for example, a luciferase, β-galactosidase, alkaline phosphatase, or green fluorescent protein reporter gene, can be used to determine transcriptional activity in screening assays according to the invention (see, for example, Goeddel (ed.), Methods Enzymol., Vol. 185, San Diego: Academic Press, Inc. (1990); see also Sambrook, supra). Accordingly, the “reporter signal” may be any kind of signal produced by the reporter genes above, which is possible to monitor.

For the culturing of cells according to the invention, the methods described in the Example section may be used, as well as any other conventionally used method.

According to the invention, strong RNA-structures have shown to give rise to both translational inhibition and potentiation, upon binding to small molecules. This is due to the fact that RNA may adopt advanced 3D-structures. If these structures are present in the 5′-UTR (5′-untranslated region), they may inhibit translation. Normally, these structures are resolved by helicases, but upon addition of molecules binding to and further stabilizing the 3D-structures, the helicases are not able to resolve these structures, which leads to inhibition of translation. Normally, binding energies <−30 kcal/mol cause complete inhibition of translation. On the other hand, one may postulate that increased expression may be caused by small molecules that stabilize translational initiation or de-stabilizes the overall stability of a large structure by its binding to a portion of it. Accordingly, the small molecules may be used as drugs affecting translation.

The strongest type of structure in RNA results from base pairing, e.g. hairpins. Binding sites for small molecules in RNA (e.g., in aptamers) are often cavities in imperfect hairpins. A list over RNA web resources related to sequences, secondary and three-dimensional structures can be found in Sühnel, J. (1997) Views of RNA on the World Wide Web. Trends in Genetics 13: 206-207. mFOLD and STAR (see below) may equally well predict strong hairpins from a given RNA-sequence. Accordingly, these hairpins may represent potential drug targets.

As said above, according to one embodiment of the invention, the molecular modeling of the RNA-structure may be performed by any one of the algoritmis of Zuker&Mathews (e.g. mFOLD) or van Batenberg (e.g. STAR).

The mFOLD algorithm (D. H. Mathews, T. C. Andre, J. Kim, D. H. Turner and M. Zuker (1998) An Updated Recursive Algorithm for RNA Secondary Structure Prediction with Improved Free Energy Parameters. American Chemical Society Symposium Series 682: 246-257; Zuker M. (2000) Calculating nucleic acid secondary structure. Current Opinion in Structural Biology 10:303-310), which is the most widely used system, is based on search for the state of minimal free energy. The mfold 3.1 software uses what are called nearest neighbor energy rules. That is, free energies are assigned to loops rather than to base pairs. These have also been called loop dependent energy rules.

The STAR (http://wwwbio.leidenuniv.nl/˜Batenburg/STAR.html; Gultyaev A. P., van Batenburg F. H. D. and Pleij C. W. A. (1995) The Computer Simulation of RNA Folding Pathways Using a Genetic Algorithm. J. Mol. Biol. 250: 37-51) is a software product, which allows predictions of secondary structures based on several algorithms. The so-called “genetic algorithm”, developed by van Batenburg, Gultyaev & Pleij (J Theor Biol 1995:174:269-280.), employs a stepwise selection of the most fit structures. The genetic algorithm simulation includes both stem formations and stem disruptions.

Moreover, the molecules to be tested for RNA-binding are added in a concentration of typically from 10 nM to 10 mM. Conventionally, most compound screening libraries contain test molecules in the molecular range of from 100 to 700 Da.

According to a second aspect of the invention, an RNA-fragment having any one of the sequences SEQ ID NO:2, SEQ ID NO:4, SEQ ID NO:6, SEQ ID NO:8, SEQ ID NO:10, SEQ ID NO:12, SEQ ID NO:14, SEQ ID NO:16 or SEQ ID NO:18, preferably any one of the sequences SEQ ID NO:1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17, is used for identifying molecules binding to the RNA-fragment in the method described above. These sequences correspond to the target region and the 5′-UTR-region of interesting RNA-fragments, i.e. fragments showing interesting structural properties.

Examples of RNA-fragments that can be used in this aspect of the invention are, for example, the ones listed below. However, the invention is not limited to these fragments, but combinations of interesting stem structures/sequences of these fragments may also be used in accordance with the invention. As a first example, the RNA fragment of C/EBP-alpha (GenBank™ Accession Number NM004364) (SEQ ID NO: 1 and 2) is disclosed, which fragment has shown an indication for diabetes. Further, DGAT (acyl CoA:diacylglycerol-acetyltransferase) (GenBank™ Accession Number NM 012079) (indication obesity) (SEQ ID NO:3 and 4), DPP-IV (dipeptidylpeptidase IV) (GenBank™ Accession Number U13710) (indication: diabetes) (SEQ ID NO:5 and 6), FABP-2 (fatty acid binding protein 2) (GenBank™ Accession Number M 18079) (indication diabetes) (SEQ ID NO:7 and 8), FATP4 (fatty acid transporter protein 4) (GenBank™ Accession Number AF055899) (indication obesity) (SEQ ID NO:9 and 10), the leptin receptor (GenBank™ Accession Number NM 002303) (indication obesity) (SEQ ID NO: 11 and 12), MyoD (GenBank™ Accession Number NM 002478) (indication obesity/cachexia) (SEQ ID NO:13 and 14), FOXC2 (GenBank™ Accession Number NM_{—}005251) (indication obesity) (SEQ ID NO:15 and 16) and SREBP-1c (serum responsive element binding protein 1 c) (GenBank™ Accession Number NM 004176) (indication diabetes/obesity) (SEQ ID NO:17 and 18) are also a part of the present invention. The indications mentioned above in relation to the specified RNA-fragments are only to be considered as examples. Other indications are also fully possible.

According to still another embodiment of the invention, the RNA-fragment used is a mouse lipoprotein lipase transcript. This fragment shows a strong 5′-UTR-structure, it has a rapid turnover of protein, and commercial antibodies are available. Moreover, it shows enzyme activity and is a blood plasma marker plasma marker. The RNA-sequence for mLPL (SEQ ID NO:19) is: 5′ GCG CCU CCU GCU CAA CCC GCU CCU GAC UGC CCC ACG CCG CGU AGU UCC AGC AGC AAA GCA GAA GGG UGC 3′ (This is the RNA sequence included in the “test assay”).

The invention will now be described by way of examples, which are included of illustrative purposes only, and are not to be seen as limiting in any respect.

### EXAMPLES OF THE INVENTION

### Example 1

The 5′-untranslated region (5′-UTR) of the human FOXC2 mRNA (SEQ ID NO:15) was used as template in an in silico secondary structure prediction. When using a structure prediction algorithm developed by van Batenberg et al., (van Batenberg, J. Theor Biol., 1995:174(3): 269-280) a strong structure evolved between positions −94 to −14 counting from the postulated initiation codon of the human FOXC2 mRNA. The calculation was performed using the STAR 4.4 software. The entire 5′-UTR sequence including the initiation codon (AUG) was included in the test sequence and the calculations were stopped after 3 iterations without changes (default setting). All variables that may be modified were used in accordance with the default settings introduced by the manufacturer. The defined RNA segment of 81 nucleotides, that was predicted to form a strong structure, is represented by the coordinates in Table IX.

### Example 2

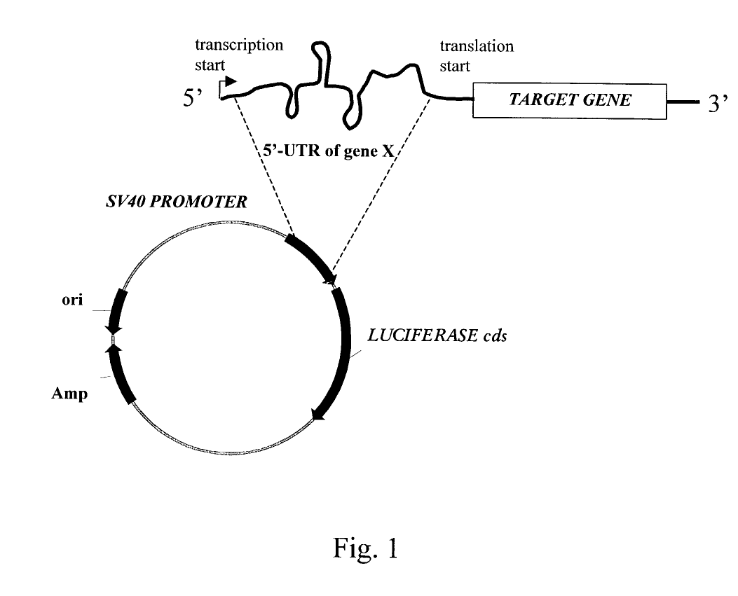

The feasibility of using a reporter-based assay is demonstrated by a pilot experiment where a 72 base pair (SEQ ID NO:19) long fragment corresponding to the mouse lipoprotein lipase transcript was inserted into a reporter vector (pGL3 control; Promega, USA). The fragment was inserted into the 5′-UTR of the luciferase gene, i.e. cells transfected with this construct expressed a transcript containing the “mLPL-structure” in the 5′-UTR (for principal vector composition, see FIG. 1). Using this construct the following transfection and assay protocol was used:

Tissue Culture

A vial of frozen cells was transferred from liquid N₂ to 37° C. to water bath until just thawed. To prevent osmotic shock and to maximize cell survival, the following was performed: 1 ml of complete medium was added to the tube. The mixture was transferred is to 15 ml tube. 10 ml of complete medium was added and gently mixed. The mix was centrifuged at 125×g for 10 minutes, whereby the supernatant was removed. The cells were resuspended in complete medium, The cells were plated at 3-5 ×10⁵ per T-75, and split every 2-3 days when they reached 70%-80% confluency. The cells were split as follows: The medium was removed, and the cells washed once with PBS. The cells were treated with 2 ml of trypsin-EDTA solution for 1-2 minutes at 37° C. 8 ml of complete medium was added. The cells were resuspended gently by pipetting. The cells were split in a ratio of up to 1:10.

Transfection

One day before transfection, the cells were trypsinized and counted, whereby the cells were plated in the complete medium at density as below (Table I).

TABLE I

| | Seeding | Volume of | | DNA | | *LF2000 |
|---|---|---|---|---|---|---|
| Culture | density | plating | | dilution | LF2000 | dilution |
| Vessel | cells/w | medium | DNA/well | volume | reagent | volume |
| 96-well | 4 × 10⁴ | 100 μl | 0.24-0.32 | 25 μl | 0.8-1.0 | 25 μl |
| | | | μg | | | |
| 24-well | 2 × 10⁵ | 500 μl | 0.8-1.0 | 50 μl | 2.5-3.5 | 50 μl |
| | | | μg | | μl | |
| 6-well | 1 × 10⁶ | 2.5 ml | 4-5 | 250 μl | 12.5-17.5 | 250 μl |
| | | | μg | | μl | |
| T-75 | 8 × 10⁶ | 20 ml | 32-40 | 1975 μl | 98-138 | 198 μl |
| | | | μg | | μl | |
| T-225 | 24 × 10⁶ | 60 ml | 95-119 | 5925 μl | 296-415 | 5925 μl |
| | | | μg | | μl | |

For a 6-well plate (when cells are 90-95% confluency, one day)

The DNA was diluted in Opti MEM I medium.

0.5-3 μg was pipetted into a tube containing 110 μl of Opti MEM I medium.

Lipofectamine 2000 reagent was diluted in Opti MEM I medium.

8 μl of Lipofectamine 2000 was pipetted into a tube containing 110 μl of Opti MEM I medium. Stable for 20 minutes.

The diluted DNA (step 1) and Lipo reagent (step 2) were combine by gentle mixing. The mix was incubated for 20 minutes at R/T. Stable for 6 hours.

The medium was removed from the wells.

The new transfection medium was added (2.5 ml/well).

The DNA-LF2000 reagent complex was added direct to each well, and gently mixed by rocking the plate back and forth.

The plates were incubated in the cell incubator for 24 hours.

Compound preparation

The compound plates were diluted from 2 mM to 410 μM. (Assume compounds will be at 10 μM at final concentration). 35 μl/well of sterile water were pipetted using the Multidrop.

5 μl of diluted compounds were transferred into 96-well assay plates or 2 μl into 384-well plates using the Robot.

Assay

The medium was aspirated in the well containing the transfected cells in the 6-well plate.

2 ml of PBS was added.

The PBS was aspirated.

1 ml of trypsin-EDTA was added.

The trypsin-EDTA was added.

50 μl of trypsin-EDTA was added.

The plates were incubated in the cell incubator for 2 minutes.

2 ml of transfect medium was added.

The cell was removed and transferred into 50 ml tube.

8 ml of transfect medium was added.

200 μl of transfected cells (4-6×10⁴ cells/well) was pipetted into 96-well plate or 80 μl of transfect cells (1.5-2×10⁴ cells/well) containing the diluted compound using the Multidrop.

The plates were incubated in the cell incubator for 24 hours.

The medium was removed using Bio-Tek plate washer.

25 or 50 μl/well Steady-Glo reagent was added using the Multidrop.

The plates were incubated at R/T for 5 minutes.

The luminescence was read with the Packard Top-Count or LJL.

The % inhibition was calculated based on controls. %I=(1-(X-BG)/(PC-BG))*100.

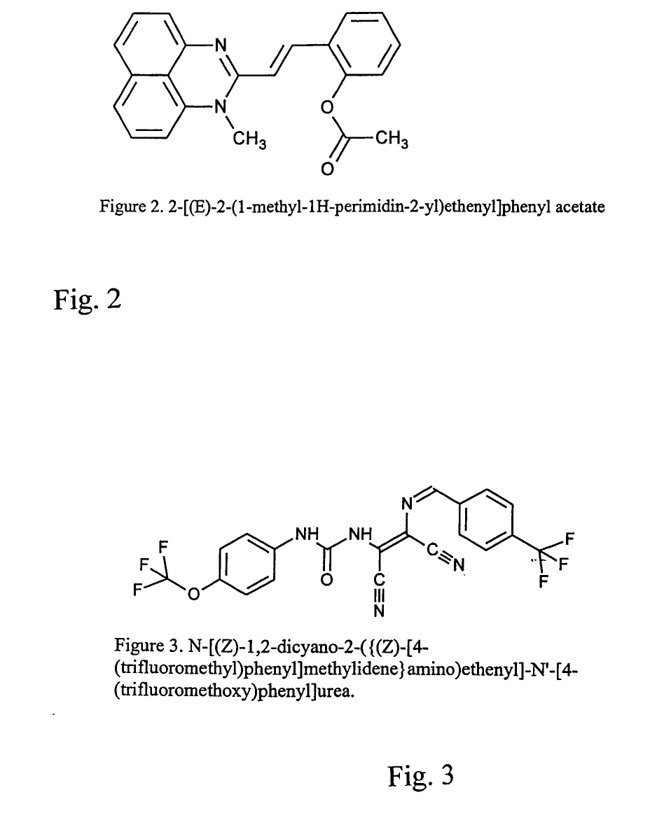

After screening 19,000 compounds, more than 900 compounds were identified that significantly affected expression of luciferase activity. In addition, a fraction (more than 30) of these compounds had significant effect on luciferase expression when the “mLPL-structure” was inserted, while no effect was observed in a control vector lacking this insert. One may therefore postulate that it is possible by such a screening procedure to identify compounds having specific effects, both activation and inhibition, mediated via a defined RNA structure. Examples of chemical entities having specific effects on mRNA having the “mLPL-structure” in its RNA are shown in FIGS. 2 and 3.

### Example 3

In Tables II to X, the first four columns indicate the positions of the stem in the sequence, counting from the 5′-end of the sequence. Columns 5-7 specify the free Gibbs energy in kcal/mol:

Column 5: gain of stacking energy

Column 6: destabilization energy of enclosed loop

Column 7: the energy that the particular stem adds to the energy of the growing structure.

Column 8 shows the stem sequences.

Column 7 indicates a sum of the energies in column 5 and 6, respectively. To calculate the total energy of a structure, all values in column 7 are added. The sum of free Gibbs energies for each substructure within the stem is a measure of its structural stability. Accordingly, a low free Gibbs energy value is a good prerequisite for a suitable drug-binding site.

TABLE II
RNA-sequence and folding coordinates for
C/EBP-alpha (SEQ ID NO:1 and 2)

| | GCGGGCGCGG GCGAGCAGGG UCUCCGGGUG GGCGGCGCGA | | | | | | |
|---|---|---|---|---|---|---|---|
| | CGCCCCGCGC AGGCUGGAGG CCGCCGAGGC UCGCCAUGCC | | | | | | |
| | GGGAGAACUC UAACUCCCCC | | | | | | |
| 1 | 2 | 3 | 4 | 5 | 6 | 7 | 8 |
| 35 | 39 | 46 | 50 | −10.8 | 3.8 | −7.0 | GCGCG |
| | | | | | | | CGCGC |
| 10 | 16 | 69 | 75 | −15.7 | 3.4 | −12.0 | GGCGAGC |
| | | | | | | | CCGCUCG |
| 19 | 27 | 54 | 62 | −16.0 | 3.7 | −12.6 | GGUCUCCGG |
| | | | | | | | CCGGAGGUC |
| 81 | 85 | 94 | 98 | −9.8 | 4.4 | −5.4 | GGGAG |
| | | | | | | | CCCUC |
| 4 | 7 | 77 | 80 | −7.8 | 2.3 | −5.5 | GGCG |
| | | | | | | | CCGU |

TABLE III
RNA-sequence and folding coordinates for DGAT
(acyl CoA:diacylglycerol-acetyltransferase)
(SEQ ID NO:3 and 4)

| GAAUGGACGA GAGAGGCGGC CGUCCAUUAG UUAGCGGCUC | | | | | | | |
|---|---|---|---|---|---|---|---|
| CGGAGCAACG CAGCCGUUGU CCUUGAGGCC GACGGGCCUG | | | | | | | |
| ACGCGGGCGG GUUGAACGCG CUGGUGAGGC GGUCACCCGG | | | | | | | |
| GCUACGGCGG CCGGCAGGGG GCAGUGGCGG CCGUUGUCUA | | | | | | | |
| GGGCCCGGAG GUGGGGCCGC GCGCCUCGGG CGCUACGAAC | | | | | | | |
| CCGGCAGGCC CACGCUUGGC UGCGGCCGGG UGCGGGCUGA | | | | | | | |
| GGCCAUG | | | | | | | |
| 1 | 2 | 3 | 4 | 5 | 6 | 7 | 8 |
| 64 | 65 | 82 | 83 | −1.5 | 3.1 | −0.2 | UG |
| | | | | | | | GC |
| 213 | 220 | 225 | 232 | −13.2 | 3.6 | −9.6 | CGCUUGGC |
| | | | | | | | GUGGGCCG |
| 207 | 211 | 234 | 238 | −10.7 | 1.0 | −9.7 | GGCCC |
| | | | | | | | UCGGG |
| 18 | 24 | 108 | 114 | −11.3 | 1.0 | −4.6 | GGCCGUC |
| | | | | | | | CUGGCGG |
| 37 | 39 | 44 | 46 | −5.1 | 3.2 | −3.6 | GCU |
| | | | | | | | CGA |
| 25 | 31 | 100 | 106 | −7.3 | 3.1 | −7.0 | CAUUAGU |
| | | | | | | | GUGGUCG |
| 162 | 166 | 174 | 178 | −12.1 | 3.3 | −8.8 | GGCCC |
| | | | | | | | CCGGG |
| 15 | 16 | 116 | 117 | −2.9 | 1.0 | −1.9 | GG |
| | | | | | | | CC |
| 181 | 184 | 190 | 193 | −8.8 | 2.0 | −6.8 | GCGC |
| | | | | | | | CGCG |
| 124 | 128 | 150 | 154 | −10.4 | 0.3 | −5.6 | ACGGC |
| | | | | | | | UGCCG |
| 130 | 133 | 145 | 148 | −7.8 | 3.1 | −7.3 | GCCG |
| | | | | | | | CGGU |
| 203 | 205 | 242 | 244 | −6.3 | 2.3 | −4.0 | GGC |
| | | | | | | | CCG |
| 34 | 36 | 97 | 99 | −5.4 | 3.8 | −2.8 | GCG |
| | | | | | | | CGC |
| 119 | 122 | 156 | 159 | −4.7 | 2.4 | −2.3 | GGGC |
| | | | | | | | UCUG |
| 134 | 135 | 141 | 142 | −3.4 | 3.0 | −2.3 | GC |
| | | | | | | | CG |
| 5 | 6 | 201 | 202 | −2.9 | 2.1 | −0.8 | GG |
| | | | | | | | CC |

TABLE IV
RNA sequence and folding coordinates for DPP-IV
(dipeptidylpeptidase IV) (SEQ ID NO:5 and 6)

| | CCCCCAGUCU CGGGCCCGAC UCUGCCCCCG UGCGCCCAGC | | | | | | |
|---|---|---|---|---|---|---|---|
| | GCCCUACACG CCCUCAGCUC GCGGGCUCCC CCGGCCGGGA | | | | | | |
| | UGCCAGUGCC GCGCCACGCG CCUCGUCCCG CCGCCUGCCC | | | | | | |
| | UGCAGCCUGC CCGCGGCGCC UUUAUACCCA GCGGCUCGGC | | | | | | |
| | GCUCACUAAU GUUUAACUCG GGGCCGAAAC UUGCCAGCCG | | | | | | |
| | AGUGACUCCA CCGCCCGGAG CAGCGUGCAG GACGCGCGUC | | | | | | |
| | UCCGCCGCCC GCGUGACUUC UGCCUGCGCU CCUUCUCUGA | | | | | | |
| | ACGCUCACUU CCGAGGAGAC GCCGACGAUG | | | | | | |
| 1 | 2 | 3 | 4 | 5 | 6 | 7 | 8 |
| 91 | 93 | 99 | 101 | −5.4 | 1.5 | −3.9 | GCG |
| | | | | | | | CGC |
| 76 | 82 | 104 | 110 | −12.3 | 3.1 | −5.9 | CGGGAUG |
| | | | | | | | GCCCUGC |
| 72 | 74 | 111 | 113 | −4.9 | 0.5 | −4.4 | CGG |
| | | | | | | | GCC |
| 85 | 86 | 102 | 103 | −1.7 | 4.2 | −0.8 | AC |
| | | | | | | | UC |
| 115 | 117 | 123 | 125 | −3.5 | 1.5 | −2.0 | CUG |
| | | | | | | | GAC |
| 38 | 43 | 158 | 163 | −13.4 | 3.5 | −5.6 | AGCGCC |
| | | | | | | | UCGCGG |
| 59 | 66 | 129 | 136 | −17.9 | 5.5 | −13.9 | UCGCGGGC |
| | | | | | | | GGCGCCCG |
| 57 | 58 | 138 | 139 | −3.4 | 2.4 | −1.5 | CC |
| | | | | | | | CG |
| 49 | 51 | 151 | 153 | −5.4 | 5.4 | −2.3 | CGC |
| | | | | | | | GCG |
| 182 | 184 | 193 | 195 | −6.3 | 4.7 | −1.6 | GGC |
| | | | | | | | CCG |
| 12 | 15 | 24 | 27 | −9.2 | 4.7 | −4.5 | GGGC |
| | | | | | | | CCCG |
| 176 | 181 | 198 | 203 | −11.0 | 3.1 | −7.9 | ACUCGG |
| | | | | | | | UGAGCC |
| 206 | 209 | 217 | 220 | −6.9 | 3.2 | −3.7 | CUCC |
| | | | | | | | GAGG |
| 30 | 35 | 223 | 228 | −9.8 | 5.3 | −4.5 | GUGCGC |
| | | | | | | | CGUGCG |
| 234 | 238 | 265 | 269 | −10.3 | 6.5 | −4.2 | GCGCG |
| | | | | | | | CGCGU |
| 243 | 245 | 251 | 253 | −5.4 | 3.8 | −2.0 | CGC |
| | | | | | | | GCG |
| 241 | 242 | 255 | 256 | −2.3 | 1.0 | −0.5 | UC |
| | | | | | | | AG |
| 274 | 278 | 295 | 299 | −7.8 | 4.9 | −2.9 | UCUCU |
| | | | | | | | AGAGG |
| 229 | 232 | 270 | 273 | −6.9 | 2.2 | −4.7 | AGGA |
| | | | | | | | UCCU |

TABLE V
RNA-sequence and folding coordinates for FABP-2
(fatty acid binding protein 2) (SEQ ID NO:7 and 8)

| GGAAUUCCAG GAGGGUGCAG CUUCCUUCUC ACCUUGAAGA | | | | | | | |
|---|---|---|---|---|---|---|---|
| AUAAUCCUAG AAAACUCACA AAAUG | | | | | | | |
| 1 | 2 | 3 | 4 | 5 | 6 | 7 | 8 |
| 9 | 14 | 21 | 26 | −8.9 | 3.7 | −5.2 | AGGAGG |
| | | | | | | | UCCUUC |

TABLE VI
RNA-sequence and folding coordinates for FATP-4
(fatty acid transporter protein 4) (SEQ ID NO:9
and 10)

| | CCCUGCUGAG ACCCGGCUCC GUGCGUCCAG GGGCGGCUAA | | | | | | |
|---|---|---|---|---|---|---|---|
| | UGCCCCUCAC GCUGUCUACG CUGCUGCAAC CGGGCCGCAU | | | | | | |
| | CUGGACGGGG CGCCGCGCGG CGAGGAACGC CGGGCCACAA | | | | | | |
| | UG | | | | | | |
| 1 | 2 | 3 | 4 | 5 | 6 | 7 | 8 |
| 62 | 67 | 97 | 102 | −11.3 | 3.1 | −6.2 | UGCUGC |
| | | | | | | | GCGGCG |
| 29 | 35 | 41 | 47 | −15.3 | 4.1 | −11.2 | AGGGGCG |
| | | | | | | | UCCCCGU |
| 23 | 26 | 49 | 52 | −7.5 | 3.7 | −3.8 | GCGU |
| | | | | | | | CGCA |
| 74 | 76 | 89 | 91 | −6.3 | 4.2 | −4.8 | GCC |
| | | | | | | | CGG |
| 71 | 73 | 93 | 95 | −4.9 | 1.0 | −3.2 | CGG |
| | | | | | | | GCC |
| 12 | 17 | 109 | 114 | −14.1 | 3.1 | −9.2 | CCCGGC |
| | | | | | | | GGGCCG |
| 18 | 20 | 104 | 106 | −5.2 | 4.2 | −2.8 | UCC |
| | | | | | | | AGG |

TABLE VII
RNA-sequence and folding coordinates for Leptin
receptor (SEQ ID NO:11 and 12)

| | GGCACGAGCC GGUCUGGCUU GGGCAGGCUG CCCGGGCCGU | | | | | | |
|---|---|---|---|---|---|---|---|
| | GGCAGGAAGC CGGAAGCAGC CGCGGCCCCA GUUCGGGAGA | | | | | | |
| | CAUGGCGGGC GUUAAAGCUC UCGUGGCAUU AUCCUUCAGU | | | | | | |
| | GGGGCUAUUG GACUGACUUU UCUUAUGCUG GGAUGUGCCU | | | | | | |
| | UAGAGGAUUA UGGGUGUACU UCUCUGAAGU AAGAUG | | | | | | |
| 1 | 2 | 3 | 4 | 5 | 6 | 7 | 8 |
| 34 | 43 | 59 | 68 | −23.8 | 3.5 | −19.4 | GGGCCGUGGC |
| | | | | | | | CCCGGCGCCG |
| 21 | 24 | 30 | 33 | −9.2 | 3.1 | −6.1 | GGGC |
| | | | | | | | CCCG |
| 49 | 50 | 56 | 57 | −3.4 | 3.9 | −0.4 | GC |
| | | | | | | | CG |
| 177 | 181 | 187 | 191 | −5.8 | 2.5 | −3.2 | UACUU |
| | | | | | | | AUGAA |
| 87 | 90 | 97 | 100 | −6.2 | 3.2 | −3.0 | GGGC |
| | | | | | | | CUCG |
| 1 | 6 | 154 | 159 | −11.7 | 6.2 | −4.7 | GGCACG |
| | | | | | | | CCGUGU |
| 81 | 85 | 101 | 105 | −5.3 | 2.4 | −3.3 | CAUCG |
| | | | | | | | GUGCU |
| 7 | 10 | 16 | 19 | −8.0 | 3.3 | −4.1 | AGCC |
| | | | | | | | UCGG |
| 112 | 115 | 121 | 124 | −5.7 | 4.0 | −2.6 | UCCU |
| | | | | | | | GGGG |
| 69 | 76 | 128 | 135 | −9.4 | 4.7 | −3.6 | CAGUUCGG |
| | | | | | | | GUCAGGUU |
| 172 | 174 | 182 | 184 | −2.8 | 3.6 | −1.6 | GGG |
| | | | | | | | CUC |
| 141 | 144 | 150 | 153 | −4.3 | 3.4 | −2.1 | UCUU |
| | | | | | | | AGGG |

TABLE VIII
RNA-sequence and folding coordinates for MyoD
(SEQ ID NO:13 and 14)

| | ACCACAAAUC AGGCCGGACA GGAGAGGGAG GGGUGGGGGA | | | | | | |
|---|---|---|---|---|---|---|---|
| | CAGUGGGUGG GGAUUCAGAC UGCCAGCACU UUGCUAUCUA | | | | | | |
| | CAGCCGGGGC UCCCGAGCGG CAGAAAGUUC CGGCCACUCU | | | | | | |
| | CUGCCGCUUG GGUUGGGCGA AAGCCAGGAC CGUGCCGCGC | | | | | | |
| | CACCGCCAGG AUAUG | | | | | | |
| 1 | 2 | 3 | 4 | 5 | 6 | 7 | 8 |
| 92 | 104 | 120 | 132 | −27.0 | 2.4 | −21.7 | CCCGAGCGGCAGA |
| | | | | | | | GGGUUCGCCGUCU |
| 46 | 50 | 60 | 64 | −6.9 | 4.2 | −2.7 | GGUGG |
| | | | | | | | CCGUC |
| 136 | 138 | 143 | 145 | −6.3 | 1.6 | −4.7 | GGC |
| | | | | | | | CCG |
| 32 | 38 | 69 | 75 | −6.5 | 3.0 | −3.5 | GGUGGGG |
| | | | | | | | UCGUUUC |
| 106 | 108 | 116 | 118 | −3.8 | 4.6 | −2.1 | AGU |
| | | | | | | | UCA |
| 43 | 44 | 66 | 67 | −1.9 | 0.7 | −1.2 | GU |
| | | | | | | | CG |
| 82 | 84 | 89 | 91 | −5.1 | 2.6 | −2.5 | AGC |
| | | | | | | | UCG |
| 12 | 14 | 154 | 156 | −6.3 | 1.0 | −1.7 | GGC |
| | | | | | | | CCG |
| 15 | 17 | 150 | 152 | −4.9 | 3.1 | −4.9 | CGG |
| | | | | | | | GCC |
| 27 | 29 | 77 | 79 | −3.8 | 2.6 | −1.7 | GGA |
| | | | | | | | UCU |
| 157 | 159 | 164 | 166 | −5.4 | 4.9 | −0.5 | GCG |
| | | | | | | | CGC |

TABLE IX
RNA-sequence and folding coordinates for FOXC2
(SEQ ID NO:15 and 16)

| | CCGCCCCUCC CGCUCCCCUC CUCUCCCCCU CUGGCUCUCU | | | | | | |
|---|---|---|---|---|---|---|---|
| | CGCGCUCUCU CGCUCUCAGG GCCCCCCUCG CUCCCCCGGC | | | | | | |
| | CGCAGUCCGU GCGCGAGGGC GCCGGCGAGC CGUCUCGGAA | | | | | | |
| | GCAGC | | | | | | |
| 1 | 2 | 3 | 4 | 5 | 6 | 7 | 8 |
| 37 | 45 | 91 | 99 | −17.6 | 4.4 | −11.3 | CUCUCGCGC |
| | | | | | | | GGGAGCGCG |
| 31 | 35 | 101 | 105 | −9.3 | 2.1 | −7.2 | CUGGC |
| | | | | | | | GGCCG |
| 60 | 63 | 78 | 81 | −9.2 | 5.2 | −5.9 | GGCC |
| | | | | | | | CCGG |
| 106 | 109 | 114 | 117 | −6.0 | 5.0 | −1.0 | CGAG |
| | | | | | | | GCUC |

TABLE X
RNA-sequence and folding coordinates for SREBP-1c
(serum responsive element binding protein 1c)
(SEQ ID NO:17 and 18)

| | UAACGAGGAA CUUUUCGCCG GCGCCGGGCC GCCUCUGAGG | | | | | | |
|---|---|---|---|---|---|---|---|
| | CCAGGGCAGG ACACGAACGC GCGGAGCGGC GGCGGCGACU | | | | | | |
| | GAGAGCCGGG GCCGCGGCGG CGCUCCCUAG GAAGGGCCGU | | | | | | |
| | ACGAGGCGGC GGGCCCGGCG GGCCUCCCGG AGGAGGCGGC | | | | | | |
| | UGCGCCAUG | | | | | | |
| 1 | 2 | 3 | 4 | 5 | 6 | 7 | 8 |
| 28 | 35 | 123 | 130 | −18.6 | 2.7 | −11.9 | GCCGCCUC |
| | | | | | | | CGGCGGAG |
| 142 | 147 | 152 | 157 | −13.2 | 4.5 | −8.7 | GCCUCC |
| | | | | | | | CGGAGG |
| 66 | 70 | 91 | 95 | −11.7 | 1.6 | −8.7 | GCGGC |
| | | | | | | | CGCCG |
| 23 | 25 | 132 | 134 | −6.3 | 1.1 | −5.2 | GCC |
| | | | | | | | CGG |
| 73 | 76 | 85 | 88 | −8.3 | 3.6 | −7.9 | CGGC |
| | | | | | | | GCCG |
| 59 | 62 | 100 | 103 | −8.8 | 2.3 | −6.3 | GCGC |
| | | | | | | | CGCG |
| 39 | 42 | 115 | 118 | −9.2 | 3.9 | −7.3 | GGCC |
| | | | | | | | CCGG |
| 49 | 51 | 104 | 106 | −5.2 | 5.2 | −0.4 | GGA |
| | | | | | | | CCU |
| 15 | 21 | 135 | 141 | −14.5 | 1.0 | −13.5 | UCGCCGG |
| | | | | | | | GGCGGCC |
| 158 | 159 | 165 | 166 | −2.9 | 1.5 | −1.4 | GG |
| | | | | | | | CC |

### Other Embodiments

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

What is claimed is:

1. A method for identifying an RNA-binding molecule, the method comprising:
(a) predicting the structure of an RNA-fragment;
(b) selecting a suitable predicted RNA-fragment of step (a), wherein the RNA-fragment comprises at least one individual stem;
(c) synthesizing a DNA-fragment corresponding to the RNA-fragment of step (b);
(d) inserting the DNA-fragment of step (c) in upstream proximity of a reporter assay gene, thereby forming a reporter construct, wherein the reporter assay gene produces a reporter signal upon translation; and
(e) performing a reporter gene assay, wherein the assay detects an interaction between a molecule to be tested for RNA-binding and the RNA-fragment of the reporter construct.

2. The method according to claim 1, wherein the at least one individual stem of the predicted RNA-fragment shows a free Gibbs energy lower than −5 kcal/mol.

3. The method according to claim 2, wherein the at least one individual stem of the predicted RNA-fragment shows a free Gibbs energy lower than −10 kcal/mol.

4. The method according to claim 1, whereby the at least one individual stem of the predicted RNA-fragment comprises less than 100 nucleotides.

5. The method according to claim 1, wherein the at least one individual stem of the predicted RNA-fragment has a ratio between number of nucleotides per base pair of less than 4.

6. The method according to claim 1, wherein the reporter gene assay is performed in living cells.

7. The method according to claim 6, wherein the reporter gene assay comprises:
(i) transfecting cells with the reporter construct;
(ii) culturing the transfected cells of step (i);
(iii) adding the molecule to be tested for RNA-binding to the cultured cells; and
(iv) monitoring the reporter signal, wherein the reporter signal indicates the interaction status between the molecule to be tested for RNA-binding and the RNA-fragment.

8. The method according to claim 1, wherein the reporter assay gene is a luciferase gene.

9. The method according to claim 1, wherein the molecule to be tested for RNA-binding is added in a concentration from 10 nM to 10 mM.

10. The method according to claim 1, wherein the RNA-fragment comprises a nucleotide sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18.

11. The method according to claim 1, wherein the RNA-fragment comprises the nucleotide sequence of SEQ ID NO:19.

12. The method according to claim 1, wherein the RNA-fragment comprises a nucleotide sequence selected from the group consisting of SEQ ID NO:1, SEQ ID NO:3, SEQ ID NO:5, SEQ ID NO:7, SEQ ID NO:9, SEQ ID NO: 11, SEQ ID NO: 13, SEQ ID NO:15, and SEQ ID NO:17.

13. The method according to claim 1, wherein the molecule to be tested for RNA-binding is a non-peptide or a non-nucleotide molecule.

14. The method according to claim 7, wherein the reporter assay gene is a luciferase gene.

15. The method according to claim 7, wherein the molecule to be tested for RNA-binding is added in a concentration from 10 nM to 10 mM.

16. The method according to claim 7, wherein the RNA-fragment comprises a nucleotide sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18.

17. The method according to claim 7, wherein the RNA-fragment comprises the nucleotide sequence of SEQ ID NO:19.

18. The method according to claim 7, wherein the RNA-fragment comprises a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, and SEQID NO:17.

19. The method according to claim 7, wherein the molecule to be tested for RNA-binding is a non-peptide or a non-nucleotide molecule.
